(19) United States
(12) Patent Application Publication
(10) Pub. No.: US 2024/0319523 A1
(43) Pub. Date: Sep. 26, 2024

(54) CONTACT LENS

(71) Applicant: LEONARDO VISION S.R.L., MONTECATINI-TERME (IT)

(72) Inventor: Leonardo PESI, MONTECATINI-TERME (IT)

(21) Appl. No.: 18/578,876

(22) PCT Filed: Jul. 13, 2022
(86) PCT No.: PCT/IT2022/050203

§ 371 Date: Jan. 12, 2024

(30) Foreign Application Priority Data
Jul. 13, 2021 (IT) 102021000018353

(51) Int. Cl. G02C 11/00; A61B 5/00; G02B 1/04

(52) U.S. Cl. CPC G02C 11/10; A61B 5/6821; G02B 1/043

(57) ABSTRACT

Contact lens made of non-hydrophilized material and including at least one functional element disposed in correspondence with a rear concave surface of the lens which is facing, during use, toward the eyeball.

### FIELD OF THE INVENTION

The present invention concerns a contact lens intended to compensate for the most common visual defects such as myopia, hyperopia, astigmatism and presbyopia, and equipped with functional elements.

### BACKGROUND OF THE INVENTION

Different types of contact lenses are currently known, essentially classified into the following two main categories:
soft contact lenses;
rigid gas permeable contact lenses (RGP).

Soft contact lenses are the most common, they adapt more easily to the cornea, remaining stable in the position of use in any condition, and are characterized by the presence, in their structure, of an aqueous component, indicatively variable between 36% and 65% by volume.

The presence of the hydrophilic material allows the soft contact lens to absorb the tear film, creating a “sponge effect”, which facilitates the passage of oxygen from the front surface of the lens to the rear surface of the lens and thus nourishes the cornea.

On the other hand, if the soft contact lens is not adequately and continuously wet by the tear film, it tends to dry out, triggering a process of dehydration and poor nourishment of the cornea.

Another disadvantage is that the dryness of these lenses makes the material less soft and produces a reduction in the value of the base radius of the lenses themselves (which are narrower), thus making them much less comfortable to wear.

The hydrophilic characteristic of soft contact lenses also entails the retention, inside the lens itself, of bacteria, viruses and various dirt, which can cause diverse and serious eye infections.

In summary, soft contact lenses can compensate for almost all visual ametropias but are particularly indicated in the case of good or excellent lacrimation and require careful cleaning.

The other category of contact lenses is represented by rigid gas permeable lenses (RGP) which have the characteristic that they are not as flexible as soft lenses and, therefore, always keep their shape once put in.

This particularity allows an excellent visual quality even in the event of corneal irregularities.

RGP contact lenses are composed of hydrophobic materials that allow to avoid the “sponge effect” that is typical of soft contact lenses, allowing the tear film, held between their rear surface and the front surface of the cornea, to have a better oxygenation of the cornea itself.

Moreover, RGP contact lenses, once put in, are not completely in contact with the corneal epithelium, but float on the surface created by the layer of the tear film, thus obtaining greater respect for the physiological environment of the eye.

Furthermore, RGP contact lenses, having a reduced wettability, are better from a hygienic point of view, since they do not absorb and do not let through the foreign substances that are deposited on their surface, reducing the risk of infection.

The main disadvantage of RGP contact lenses, however, is that they are made of a rigid material, and this leads to less tolerability and comfort, especially at the beginning of their use, compared to soft contact lenses.

In summary, RGP contact lenses can compensate for almost all visual ametropias, they are indicated in the case of irregular cornea and even in cases of medium lacrimation, but are not very comfortable.

In addition to the aspects mentioned above, which are specific to the specific sector of contact lenses, it is also known in a more general way, in the field of medicine, that the continuous monitoring of physiological parameters, such as for example the level of glucose, the heartbeat or suchlike, is of increasing interest, as well as the possibility of continuously treating pathologies by administering drugs or medical compounds in a continuous, slow-release manner, or at predefined time intervals.

In particular, it is known that information relating to physiological parameters such as those reported above can also be obtained by analyzing the tear film or by contact with the ocular surface, while the administration of drugs through the eye, by means of eye drops, for example, is also known in the state of the art.

Patent application US-A1-2017/020441 describes a contact lens with integrated sensors configured to detect some physiological parameters in the eyeball and/or in the tear film. These sensors operate through piezoelectric transducers located on the surface of the lens facing toward the outside when the lens is installed on an eyeball. The detections are performed through ultrasonic pulses sent to the cornea of the eye. The lens does not include any sensor elements in contact with the tear film and/or the eyeball.

Patent application US-A1-2017/042480 describes a lens comprising a detection and analysis device comprising, among other components, an electrochemical sensor. The device as a whole, and in particular the sensor, are incorporated inside the thickness of the lens.

Known lenses do not allow to continuously monitor certain physiological parameters, or to continuously treat specific pathologies, and therefore it is necessary to use external devices to be applied to the eye.

There is therefore a need to perfect a contact lens which can overcome at least one of the disadvantages of the state of the art.

In order to do this, it is necessary to solve the technical problem of providing a contact lens that houses functional elements suitable to carry out at least one or both of the above functions of continuous monitoring and/or administration through the eyeball of drugs or other liquids, such as for example artificial tears or suchlike.

Another purpose of the present invention is to provide a contact lens which is similar to the currently known soft contact lenses, so as to adapt to the shape of the cornea, improving tolerability and comfort of use, in any condition.

Another purpose is to provide a non-hydrophilized contact lens which, once put in, allows the tear film to remain constantly inside the cavity that is created between the cornea and the lens itself, without being absorbed (no “sponge effect”), in a similar but more optimal manner than what happens with known RGP contact lenses, keeping lacrimation in its natural state.

Yet another purpose is to provide a contact lens which, once put in, allows the tear film to move freely between the cornea and the lens, at least during eye movements or blinking of the eyelids.

The Applicant has devised, tested and embodied the present invention to overcome the shortcomings of the state of the art and to obtain these and other purposes and advantages.

### SUMMARY OF THE INVENTION

The present invention is set forth and characterized in the independent claim. The dependent claims describe other characteristics of the present invention or variants to the main inventive idea.

In accordance with the above purposes and in order to resolve the technical problem disclosed above in a new and original way, also achieving considerable advantages compared to the state of the prior art, a lens according to the present invention is made of non-hydrophilized material and comprises at least one functional element disposed in correspondence with its rear surface and protruding with respect to the latter so as to be facing, during use, toward the eyeball. The functional element is configured as a sensor suitable to detect the presence of one or more physiological parameters, or as a reservoir suitable to deliver one or more liquids for treating the eye onto the corneal surface.

Particularly advantageously, the lens comprises, in correspondence with its rear surface, a plurality of protuberances configured to at least partly contain a functional element.

Preferably, the lens comprises an internal sector and an external sector, the latter extending annularly outside the internal sector. The sectors both have an own rear surface facing, during use, toward the eyeball of the user. In particular, the internal sector is substantially circular in shape. The external sector is advantageously substantially annular in shape.

According to one aspect of the present invention, the above said at least one functional element is disposed in the external sector.

In accordance with another aspect of the present invention, the lens comprises a plurality of functional elements, disposed homogeneously distributed in correspondence with the rear surface of the external sector, that is, the surface destined to contact the eyeball, each one of the functional elements being disposed longitudinally parallel to a respective radial directrix exiting from the center of the lens.

In accordance with some embodiments, the internal sector and the external sector have different radii of curvature, measured radially with respect to the lens. Preferably, the radius of curvature of the external sector is larger than the radius of curvature of the internal sector. This ensures that the external sector adheres to the eyeball completely and correctly. The internal sector and the external sector are preferably circular in shape and concentric with each other and with respect to the center of the lens.

The internal sector is configured to be disposed on the cornea while the external sector is configured to be disposed on the sclera, outside the iris.

In accordance with some embodiments of the invention, the lens also comprises an intermediate sector located between the internal sector and the external sector, and concentric with them. Advantageously, the intermediate sector has a radius of curvature, measured radially with respect to the lens, which is different from the radii of curvature of the internal sector and of the external sector.

According to some embodiments, the lens also comprises a connection device for connecting the at least one functional element to a communication device. The connection device is incorporated in the thickness of the lens itself, in such a way as to be intersected by at least one functional element. By doing so, the functional elements can be connected to the connection device in a simple and reliable manner.

The communication device is configured to communicate the parameters detected by the sensors to an external device, for example a computer or suchlike. The communication device, for example an antenna, is connected to the connection device, or is part of the connection device.

The connection device can comprise a support element and one or more electrically conductive elements connected to the support element and also to the communication device, in order to communicate the data received from the sensors. Advantageously, the support element and the one or more electrically conductive elements have a circular shape with different diameters from each other, and they are disposed coaxial with each other and with respect to a central axis of the lens, extending for 360° around this axis so as to reciprocally connect the functional elements. In this way, the connection device can be contained in a sector of the lens, for example in the external sector described above.

In other embodiments, the electrically conductive elements can have the most varied geometries. For example, they can extend only for one circular sector, and/or conductive elements with different diameters that extend over different circular sectors, or they can develop as broken or curved lines, open or closed, therefore respectively only on one portion of the lens or for 360°.

If the functional element is configured as a sensor, it comprises a body with a predefined shape that protrudes from the rear surface of the lens, and at least one electrically conductive sensor element associated with such body. Preferably, the sensor elements are partly contained inside the protuberances defined by the body. The body is therefore suitable to attach the sensor element to the lens and to put the sensor element in contact with the eyeball and/or with the tear film, or at least in the conditions to allow the detection of physiological parameters. Preferably, the body is configured as a protuberance, advantageously made in one piece with the lens. More preferably, it is configured to be in contact at least with the tear film when the lens is installed on an eye. The sensor element is at least partly immersed, that is, incorporated or wrapped, in the body.

Advantageously, the sensor comprises at least one terminal partly immersed in the body and electrically connected to the sensor element, so as to receive and convey an electrical signal emitted by the sensor element. The terminal is electrically conductive and is suitable to be electrically connected to the connection device, preferably to one of the one or more electrically conductive elements.

According to some embodiments, the sensor comprises a plurality of terminals, the number of which is equal to the number of electrically conductive elements of the connection device. Advantageously, each terminal is configured to be electrically connected to a respective electrically conductive element. In this way, it is possible to configure the sensor so as to have different terminals, each one configured to detect a respective parameter and to be connected to a predefined electrically conductive element in order to specifically convey the data detected by each terminal.

The sensor also preferably comprises at least one removable joining member suitable to electrically connect the at least one terminal to the connection device. The joining member can be configured to connect in a removable way to the at least one terminal. For example, the at least one terminal can comprise a connection seating, and the connection portion of the joining member is configured to be inserted into the connection seating. For this purpose, the joining member can comprise a connection portion configured to be inserted into the connection seating in a removable manner.

According to some embodiments, if the functional element is configured as a reservoir containing a liquid to be delivered onto the corneal surface, it comprises a body in which a containing space for the liquid is made. Advantageously, the body can be configured as a protuberance, preferably made in one piece with the lens. It can be provided that a spongy matrix is disposed in the containing space to retain the liquid, so as to ensure its gradual delivery over time. In addition or alternatively, it can be provided that the reservoir comprises a lid suitable to close the containing space and equipped with a plurality of through holes, of micrometric size, so as to allow the liquid contained in the containing space to flow out. In one variant, each of the holes can be provided with a closing element which can be selectively opened in order to control the outflow of the liquid through the through holes.

In accordance with some embodiments, the lens also comprises a plurality of micro-protuberances facing, during use, toward the eyeball, which allow to raise the lens with respect to the corneal surface by a few micrometers. These micro-protuberances, different from the protuberances indicated above, are located in the internal sector of the lens, more preferably in a peripheral zone of the internal sector of the lens. This raising of the lens with respect to the corneal surface advantageously allows a better oxygenation of the eye, allowing the formation of circulation micro-channels that facilitate the movement of the tear fluid between the surface of the eye and the rear surface of the lens, and/or allowing a better movement of the lens on the eye, particularly for lenses with a small thickness.

### DESCRIPTION OF THE DRAWINGS

These and other aspects, characteristics and advantages of the present invention will become apparent from the following description of some embodiments, given as a non-restrictive example with reference to the attached drawings wherein:

FIG. 1 is a front view of a contact lens according to the invention;

FIG. 1A is an enlargement of a portion of the lens of FIG. 1;

FIG. 2 is a lateral view of the lens of FIG. 1;

FIG. 3 is a front view of a connection device of the lens of FIG. 1;

FIGS. 4A, 4B and 4C are plan views of three functional elements according to three alternative embodiments described here;

FIG. 5 is a three-dimensional view of a functional element connected to the connection element;

FIG. 6 is an enlarged detail of FIG. 5;

FIGS. 7 and 8 are a three-dimensional view and a longitudinal section view, respectively, of a second type of functional element of the lens, taken along the line VIII-VIII of FIG. 7;

FIGS. 9 and 10 are a three-dimensional view and a longitudinal section view, respectively, of a variant of the second type of functional element of the lens in accordance with some embodiments described here, taken along the line X-X of FIG. 9;

FIG. 11 is a partial and schematic section view of a portion of the lens; and

FIG. 11A is an enlarged detail of FIG. 11.

It is to be clarified that in the present description the phraseology and terminology used, as well as the figures in the attached drawings also as described, have the sole function of better illustrating and explaining the present invention, their function being to provide a non-limiting example of the invention itself, since the scope of protection is defined by the claims.

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS OF THE PRESENT INVENTION

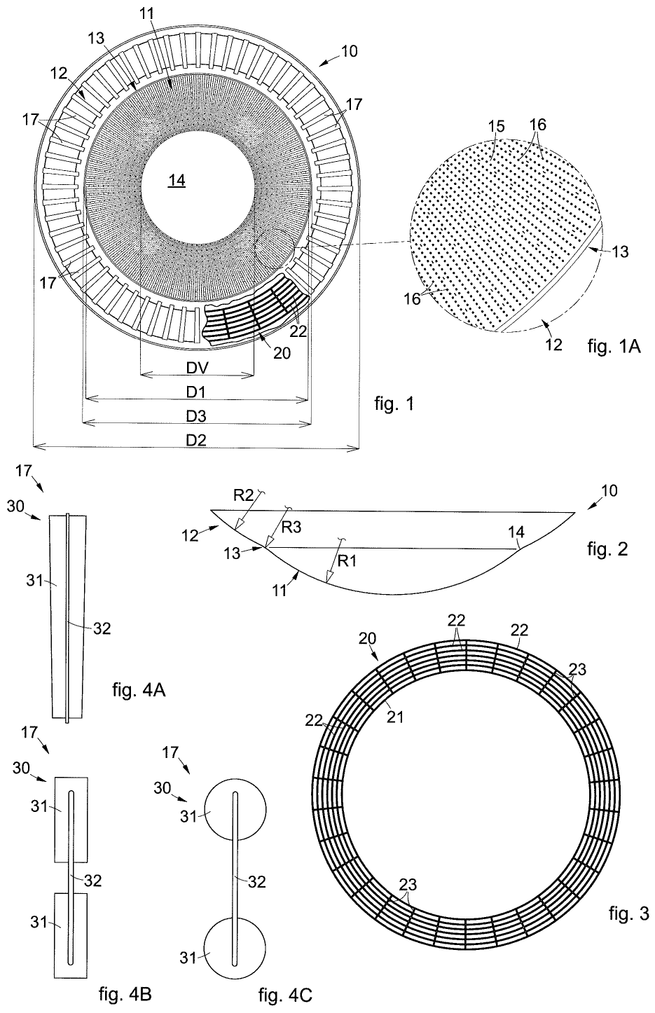

With reference to FIG. 1, a contact lens 10 according to the present invention, hereafter also just lens 10, comprises an internal sector 11 and an external sector 12 located outside the sector 11 and concentric therewith. The internal sector 11 has a circular shape while the external sector 12 has an annular shape.

The lens 10, as well as the sectors 11, 12, has a concave shape and has a rear surface facing the eyeball during use. Please note that the rear surface of the lens 10 is defined by the sum of the surfaces of the sectors 11, 12.

The internal sector 11 is configured to be disposed on the pupil and on the iris, and for this purpose it has an external diameter D1 comprised between 11.50 mm and 12.50 mm. The external sector 12 is instead configured to be disposed on the sclera, and has an external diameter D2 comprised between 13.50 mm and 16 mm.

In FIGS. 1 and 2, the lens 10 also comprises an intermediate sector 13 with an annular shape and located between the sectors 11 and 12, concentrically therewith. The intermediate sector 13 has an external diameter D3 comprised between 12.50 mm and 13.50 mm, and an internal diameter equal to the diameter D1 of the internal sector 11. Similarly, the external sector 12 has an internal diameter which corresponds to the external diameter D3 of the intermediate sector 13, or to the diameter D1 of the internal sector 11, if the intermediate sector 13 is not provided.

The sectors 11, 12 and 13 are made in a single body and each one has its own radius of curvature R1, R2, R3, different from the others. By radius of curvature we mean the radius of curvature measured radially with respect to the lens 10 itself (FIG. 2).

In particular, the sector 11 has the smallest radius of curvature R1 of the three sectors, so that the internal sector 11 has a more pronounced camber so as to appropriately correct the visual defect. The radius of curvature R1 of the internal sector 11 can be comprised between 6.50 mm and 9.50 mm.

The radius of curvature R2 of the sector 12 is larger than the radius of curvature R1 of the internal sector 11, in particular it is larger than it by a value comprised between 1 mm and 6 mm (R1+1 mm<R2<R1+6 mm), preferably equal to about 4 mm.

The radius of curvature R3 of the sector 13 is instead larger than the radius of curvature R2 of the external sector 12, and therefore also of the radius of curvature R1 of the internal sector 11. For example, the radius of curvature R3 is larger than R1 by a value comprised between 2 mm and 8 mm (R1+2 mm<R3<R1+8 mm), preferably equal to about 6 mm.

The internal sector 11 is divided into an internal zone 14 which extends within a visual diameter Dv smaller than the diameter D1, preferably corresponding to the diameter of the cornea. The visual diameter Dv, in fact, is preferably identified by the maximum size of the projection of the contour corresponding to the maximum average diurnal dilation of the pupil on the rear surface, that is, the surface facing the eyeball, of the contact lens 10.

The visual diameter Dv has a maximum value approximately equal to 8 mm, and a typical preferential value thereof is between 5 and 6 mm.

The internal sector 11 also comprises a peripheral zone 15, completely external to the internal zone 14 and, therefore, completely external to the visual diameter Dv, and which extends, at most, up to the external limit of the internal sector 11. The peripheral zone 15, annular in shape, extends between the visual diameter Dv and the diameter D1 and it is configured as a natural continuation of the internal zone 14; for this reason, the peripheral zone 15 also generally has a concave shape. The internal zone 14 and the peripheral zone 15 preferably have the same radius of curvature.

According to one variant, which can be seen in the enlargement of FIG. 1A, the contact lens 10 comprises a plurality of micro-protuberances 16, which protrude from the rear surface of the lens 10 in such a way as to be facing, during use, toward the eyeball.

According to some embodiments described here, the micro-protuberances 16, which can also be called “pillars” or “micro-pillars”, are made in the internal sector 11, preferably in the peripheral zone 15.

According to a preferential embodiment, the micro-protuberances 16 are provided on the entire rear surface of the peripheral zone 15 and are disposed starting from the visual diameter Dv up to the diameter D1 of the internal sector 11.

Preferably, the micro-protuberances 16 are homogeneously distributed on the rear surface of the lens 10, in particular on the entire surface of the peripheral zone 15, for example according to a regular geometric pattern defined by a plurality of rows radiating from a central axis X of the contact lens 10.

The micro-protuberances 16 can have a cylindrical, truncated cone or paraboloid, symmetrical or asymmetrical, shape. If of a cylindrical shape, the micro-protuberances 16 have transverse sizes (that is, diameters) comprised between 1 μm and 500 μm, preferably between 20 μm and 300 μm. The same values are also applicable if the micro-protuberances are of a different shape, in this case constituting their maximum overall transverse size.

The micro-protuberances 16 can have a constant or variable height. Furthermore, there can be provided micro-protuberances 16 of different heights in different zones of the lens 10.

For example, the micro-protuberances 16 have a maximum height comprised between 5 μm and 25 μm, preferably equal to 10 μm, which substantially corresponds to the thickness of the tear film, usually comprised between 8.5 μm and 9.5 μm.

The distance between the respective centers of two adjacent micro-protuberances 16 can be comprised between 30 μm and 500 μm, preferably between 60 μm and 140 μm, more preferably equal to about 100 μm. This distance can be comprised between one and four times the maximum height of the micro-protuberances 16, preferably being comprised between one and three times such height.

The lateral walls of the micro-protuberances 16 are oriented, with respect to the rear surface of the lens 10, in such a way as to form an angle comprised between 80° and 100° with the latter, preferably between 85° and 95°, even more preferably an angle of substantially 90°.

Advantageously, the number of micro-protuberances 16 can vary between 300 and 65,000, preferably between 2,000 and 20,000, more preferably between 5,000 and 8,000, thus ensuring good oxygenation of the eye.

In addition to the micro-protuberances 16, the contact lens 10 comprises a plurality of functional elements 17 located in correspondence with the external sector 12, in particular on its rear surface, in order to be in contact with the eyeball (FIG. 1). These functional elements 10 are electrically connected to a connection device 20 (FIG. 3) which is advantageously immersed inside the body of the lens 10 (FIGS. 1 and 4). Since the functional elements 17 are disposed only in the external sector 12, it is advantageous to provide that the connection device 20 also extends only in the external sector 12.

The functional elements 17 are present in a number comprised between 2 and 340, the number of which is correlated to their sizes, as well as to the sizes of the lens 10.

The functional elements 17 can be configured in various ways, for example as sensors, in particular configured to detect the presence of a predetermined physiological compound in the tear fluid, such as glucose, pH, heart rate, ocular pressure, or any another parameter of biological interest whatsoever, or as a reservoir containing a liquid of interest, for example artificial tears or medical liquids or eye drops, which is gradually released into the eye in order to carry out the slow-release treatment provided.

The connection device 20 is configured to connect the functional elements 17 to a communication device, for example an antenna or a control device, so as to be able to communicate data detected by the functional elements 17, if these operate as sensors, or to control them remotely.

For this purpose, the connection device 20 comprises an annular support element 21 and a plurality of conductive elements 22, in this case seven, but their number must not be construed as limited to this, which are also annular, and each have a respective diameter comprised between D3 and D2 (FIG. 3). By conductive element we mean an electrically conductive element.

The support element 21 and the conductive elements 22 are located concentrically with each other and with respect to the center of the lens 10; the support element 21 being inside the conductive elements 22 and connected to them by means of a plurality of rods 23 disposed in a radial pattern and homogeneously distributed around the support element 21 (FIG. 3).

The communication or control device is preferably integrated with the support element 21, but it can also be attached to any one of either the support element 21 or the conductive elements 22. Please note that the latter have a circumference equal to 360°, this allows them to selectively connect different functional elements 17 of the lens 10 together. The way in which the functional elements 17 are connected to the connection device 20 will be explained in detail below.

With reference to FIGS. 4A, 4B, 4C, 5 and 6, embodiments in which the functional element 17 is configured as a sensor 30 are described. In a first variant, the sensor 30 comprises a single body 31, formed by a protuberance, and a sensor element 32 that crosses it longitudinally in correspondence with one of its surfaces (FIG. 4A). The sensor element 32 is suitably made of electrically conductive material, so as to transmit an electrical signal following the detection of a compound of interest. The sensor element 32 can be, for example, a functionalized electrode. In this variant, the body 31 of the sensor 30 has a parallelepiped shape, with a length comprised between 700 μm and 2000 μm, preferably equal to about 1400 μm, with a width comprised between 160 μm and 900 μm, preferably equal to about 500 μm, and a height comprised between 5 μm and 25 μm, preferably equal to about 15 μm

This body, during use, protrudes from the rear surface of the lens 10, more precisely from the rear surface of the external sector 12 of the lens 10, defining a functional element in relief.

It is also possible to provide a section with increased sizes in correspondence with the surface of the body 31 which, during use, is in contact with the lens 10. In this section, the length and width are increased by 20 μm with respect to the rest of the body 31, while the height is that indicated above.

FIG. 4B shows a variant of the sensor 30 in which the body 31 is divided into two separate parts, both parallelepiped in shape, and connected to each other by means of the sensor element 32. The division of the body 31 into two separate parts allows to keep a certain flexibility of the lens 10 in correspondence with the functional element 17, allowing the sector 12 to better adhere to the eyeball. In this embodiment, each part of the body 31 has, by way of example, a length comprised between 350 μm and 1000 μm, preferably equal to about 700 μm, a width comprised between 80 μm and 450 μm, preferably equal to about 250 μm, and a height comprised between 5 μm and 25 μm, for example equal to about 15 μm.

FIG. 4C shows another variant in which the body 31 is divided into two parts that are separate but connected by means of the sensor element 32, the two parts being however circular in shape. Please note that the two parts of the body 31 of the sensor 30 can have any shape whatsoever, according to requirements.

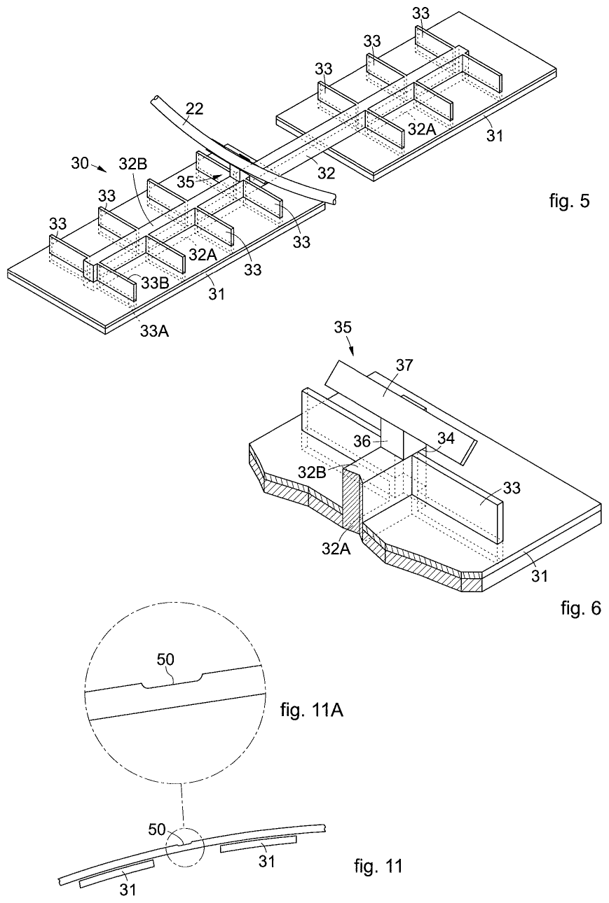

The mode of connection of the sensor 30 to the connection device 20 is shown in detail in FIG. 5, in which the sensor 30 is of the type shown in FIG. 4B.

The sensor 30 comprises a plurality of terminals 33 configured as plates partly immersed in the body 31 of the sensor 30, each plate comprising an insertion seating 34 for the insertion of a joining member 35 (FIGS. 5 and 6). The plates are disposed perpendicular with respect to the sensor element 32 and contained in a plane perpendicular to the body 31 of the sensor 30.

In the example shown, the terminals are seven in number, and are placed in such a way that each one is at the height of a respective conductive element 22 of the connection device 20. FIG. 5 shows only one conductive element 22, but during use all the conductive elements 22 above the sensor 30 are present.

As previously stated, joining members 35 are provided to be inserted into the insertion seating 34 (FIG. 6) of the terminals 33, so as to connect them to a respective conductive element 22 of the connection device 20 (FIG. 5). Each joining member has a support rod 36 to which a contact portion 37 is perpendicularly attached, thus forming a “T” (FIG. 6). The contact portion 37 is intended to come into contact with a respective conductive element 22 of the connection device 20. Advantageously, the support rod 36 has a cross section corresponding in shape to the cross section of the seating 34, or it comprises a connection portion with a cross section corresponding in shape to the cross section of the seating 34, so as to make the connection by means of a same-shape coupling.

Please note that the sensor element 32 develops longitudinally along the sensor 30 in such a way as to connect all the terminals 33 together. In particular, the sensor element connects the terminals 33 of a same part of the body 31 by means of a first segment 32A located in the surface of each part of the body 31 which is then facing toward the eyeball (FIG. 5). The first segment 32A of the sensor element 32 connects to a first longitudinal edge 33A of each of the terminals 33 of the same part of the body 31 in which it is located. The first longitudinal edge 33A is advantageously also located in the surface of the body 31 which is facing toward the eyeball. Alternatively, the first segment 32A of the sensor element 32 and/or the first longitudinal edge 33A of the terminals are incorporated in the body 31, or only part of the terminals 33 have their first longitudinal edge 33 in correspondence with the surface of the body 31.

There is a second segment 32B of the sensor element that connects all seven terminals 33 of the sensor 30 to each other, in correspondence with a second longitudinal edge 33B thereof, opposite to the first (FIGS. 5 and 6). In particular, the second edge 33B of the terminals 33 is distanced from the body 31 toward the connection device 20. Preferably, the segments 32A, 32B of the sensor element 32 connect to the terminals 33 in correspondence with the insertion seating 34, which extends between the two longitudinal edges 33A, 33B of the plate, so as to improve the transmission of the electrical signal toward the connection device 20 through the joining member 35.

In particular, the presence of the terminals 33, each facing a corresponding conductive element 22, allows to choose which conductive element 22 to connect each of the sensors 30 to. It is also possible to choose not to connect a sensor 30 to any of the conductive elements 22 of the connection device 20, providing that no joining member 35 is inserted into any of the terminals 33.

In this way, it is possible to provide that the lens 10 has seven different sensors capable of detecting seven different parameters, or that there are seven different types of functional elements 17 in a same lens 10, each having a sensor 30 able to detect a dedicated physiological parameter.

With reference to FIGS. from 7 to 10, a second type of functional element 17 is described, which is configured as a reservoir 40 containing a liquid L to be delivered, for example an eye drop, artificial tears, or similar compositions.

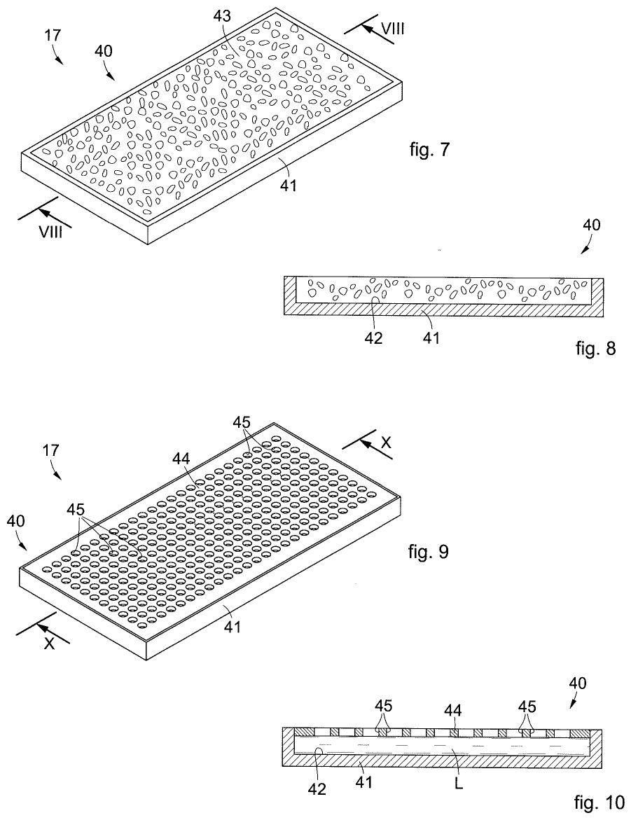

FIGS. 7 and 8 show a first variant in which the reservoir 40 comprises a body 41, similar to the body 31 of the sensor 30, in which a containing space 42 is made to contain the fluid. The containing space 42 can be open in correspondence with a surface of the body 41, for example the surface that during use comes into contact with the eyeball.

In the first variant of the reservoir 40, the liquid is contained in a spongy matrix 43 which is inserted into the containing space 42, preferably in such a way as to fill it completely (FIGS. 7 and 8). The delivery occurs gradually through the diffusion of the liquid L into the tear fluid that covers the corneal surface.

FIGS. 9 and 10 show a second variant of the reservoir 40, which does not comprise a spongy matrix, but a lid 44 to close the containing space 42 equipped with through holes 45, in particular of micrometric size, for the outflow of the liquid.

Advantageously, the through holes 45 are equipped with respective closing elements (not shown) which are able be driven remotely, or automatically, in order to allow to open and close each of the through holes 45 according to requirements. It can for example be provided that the closing elements are configured as membranes suitable to selectively allow the passage of a few drops of liquid, if certain conditions of use occur. Alternatively, a programmable control unit can be provided that drives one or more of the closing elements in order to control the release of the liquid L over time. The drives can be transmitted to the reservoir 40 by means of the connection device 20.

The flexibility of the external sector 12 is an advantage in that it allows to keep all or part of the external sector 12 adhering to the eyeball, which allows the sensors 30 to more effectively detect the physiological parameters, and the reservoirs 40 to gradually release the substance of interest. The division of the body 31 of the sensor 30 into two or more parts increases the flexibility of the external sector 12.

To further increase the flexibility of the lens 10 in correspondence with the external sector 12, it is possible to make a groove 50 on the external surface of the lens 10, in correspondence with the space comprised between the two parts of the body 31 (FIG. 11). The groove 50 advantageously has a depth smaller than the thickness of the lens 10 in the same point, so as not to excessively weaken the structure of the lens. For example, it can be provided that the groove 50 has a width comprised between 60 and 300 μm, for example 150 μm, and a depth comprised between 5 and 60 μm, for example 30 μm.

It is clear that modifications and/or additions of parts may be made to the contact lens as described heretofore, without departing from the field and scope of the present invention, as defined by the claims.

It is also clear that, although the present invention has been described with reference to some specific examples, a person of skill in the art shall certainly be able to achieve many other equivalent forms of contact lenses, having the characteristics as set forth in the claims and hence all coming within the field of protection defined thereby.

1. Contact lens made of non-hydrophilized material, comprising at least one functional element disposed in correspondence with a rear surface of said lens and protruding with respect to said surface so as to be facing, during use, toward the eyeball, being configured as a sensor suitable to detect the presence of one or more physiological parameters, or as a reservoir suitable to deliver, onto the corneal surface, one or more liquids for treating the eye.

2. Lens as in claim 1, further comprising a circular shaped internal sector and an annular shaped external sector which extends annularly outside said internal sector, and said at least one functional element is disposed in said external sector.

3. Lens as in claim 2, further comprising a plurality of functional elements homogeneously distributed in correspondence with the rear surface of said sector intended to contact the eyeball, each being disposed longitudinally parallel to a respective radial directrix exiting from the center of said lens.

4. Lens as in claim 2, wherein said sector has a first radius of curvature smaller than a second radius of curvature of said sector.

5. Lens as in claim 1, further comprising a connection device for connecting said at least one functional element to a communication device, incorporated in the thickness of said lens so as to intersect said at least one functional element, and said connection device comprises an annular support element and a plurality of electrically conductive elements connected to said support element and to said communication device in order to communicate the data received from said sensors.

6. Lens as in claim 5, wherein said sensor comprises at least one body protruding from the rear surface of said lens and an electrically conductive sensor element associated with said body.

7. Lens as in claim 6, wherein said at least one body is configured as a protuberance made in one piece with the lens.

8. Lens as in claim 6, wherein said sensor element is at least partly immersed in said at least one body.

9. Lens as in claim 6, wherein said sensor comprises at least one electrically conductive terminal configured to be electrically connected to one of said plurality of electrically conductive elements of said connection device, said at least one terminal being partly immersed in said body and electrically connected to said sensor element.

10. Lens as in claim 9, wherein said sensor comprises a plurality of terminals, the number of which is equal to the number of electrically conductive elements, wherein each one of said plurality of terminals is configured to be electrically connected to a respective electrically conductive element.

11. Lens as in claim 9, wherein said sensor comprises at least one removable joining member suitable to electrically connect said at least one terminal to said connection device.

12. Lens as in claim 11, wherein each one of said plurality of terminals comprises a respective connection seating, and in that said joining member is configured to be inserted into said connection seating.

13. Lens as in claim 1, wherein said reservoir comprises a body in which a containing space for the liquid to be delivered is made, provided with a lid equipped with a plurality of through holes, and/or in that said reservoir comprises a spongy matrix disposed inside said containing space and suitable to contain a liquid to be delivered.

14. Lens as in claim 13, wherein said body is configured as a protuberance made in one piece with the lens

15. Lens as in claim 1, further comprising a plurality of micro-protuberances, which protrude from the rear surface of said lens so as to be facing, during use, toward the eyeball and which are homogeneously distributed in a peripheral zone of said internal sector, according to a regular geometric pattern defined by a plurality of rows radiating from a central axis of said lens.

16. Lens as in claim 1, further comprising a groove on an external surface.

17. Lens as in claim 16, wherein said sensor comprises at least one body protruding from the rear surface of said lens and an electrically conductive sensor element associated with said body.

18. Lens as in claim 17, wherein said groove is in correspondence with a space comprised between two parts of the body.

19. Lens as in claim 18, wherein said groove has a depth smaller than the thickness of the lens, wherein said groove has a width between 60 μm and 300 μm, and a depth between 5 μm and 60 μm.

20. Lens as in claim 16, wherein said groove has a depth smaller than the thickness of the lens, wherein said groove has a width between 60 μm and 300 μm, and a depth between 5 μm and 60 μm.
